(19) United States
(12) Patent Application Publication
(10) Pub. No.: US 2025/0380953 A1
(43) Pub. Date: Dec. 18, 2025

(54) SELF-RETAINING GLENOID REAMER

(71) Applicant: Zimmer, Inc., Warsaw, IN (US)

(72) Inventors: Robert E. Montgomery, South Bend, IN (US); Peter Darrigan, Fort Wayne, IN (US)

(21) Appl. No.: 19/187,056

(22) Filed: Apr. 23, 2025

Related U.S. Application Data
(60) Provisional application No. 63/659,087, filed on Jun. 12, 2024.

(51) Int. Cl. A61B 17/16; A61B 17/00

(52) U.S. Cl. CPC A61B 17/1617; A61B 2017/00477

(57) ABSTRACT

A surgical reamer assembly can include a reamer head configured to ream a bone of a patient. The reamer head can include an internal hex housing. A ball hex driver can be insertable into the internal hex housing. The surgical reamer assembly can also include a retaining ring located within the internal hex housing. The retaining ring can be configured to retain the ball hex driver within the reamer head.

### CLAIM OF PRIORITY

This application claims the benefit of U.S. Provisional Patent Application Serial No. 63/659,087, filed on June 12, 2024, the benefit of priority of which is claimed hereby, and which is incorporated by reference herein in its entirety.

### TECHNICAL FIELD

Examples described herein generally relate to a reamer and, more specifically, to a self-retaining glenoid reamer.

### BACKGROUND

Reamers are specialized surgical tools designed to precisely shape and prepare bone cavities during orthopedic procedures, such as joint replacements. Reamers can ensure the cavities conform accurately to the dimensions required for implant components, enhancing prosthetic device stability and fit. Surgical reamers must be meticulously crafted to remove bone tissue efficiently while minimizing trauma to the surrounding areas, which helps maintain the integrity of the bone structure and facilitates the healing process.

### SUMMARY

In examples, a surgical reamer assembly can include a reamer head configured to ream a bone of a patient. The reamer head can include an internal hex housing. A ball hex driver can be insertable into the internal hex housing. The surgical reamer assembly can also include a retaining ring located within the internal hex housing. The retaining ring can be configured to retain the ball hex driver within the reamer head.

In examples, a surgical reamer assembly can include a reamer head and a retaining ring. The reamer head can include an internal hex housing, the internal hex housing configured to receive a ball hex driver. The retaining ring can be located within the internal hex housing. The retaining ring can be configured to expand and contract to permit the insertion and removal of the ball hex driver from the reamer head and retain the ball hex driver within the reamer head, respectively.

### BRIEF DESCRIPTION OF THE DRAWINGS

Various examples are illustrated in the figures of the accompanying drawings. Such examples are demonstrative and not intended to be exhaustive or exclusive examples of the present subject matter.

FIG. 1 is a perspective view of an example of a surgical reamer assembly.

FIG. 2 is an enlarged perspective view of a portion of an example of a surgical reamer assembly.

FIG. 3 is an enlarged cross-sectional view of a portion of an example of a surgical reamer assembly.

FIG. 4 is an enlarged cross-sectional view of a portion of an example of a surgical reamer assembly.

FIG. 5 is a schematic illustration of an example removal tool and an example retaining ring.

FIG. 6 is an enlarged perspective view of a portion of an example of a surgical reamer assembly.

FIG. 7 is an enlarged cross-sectional view of a portion of an example of a surgical reamer assembly.

FIG. 8 is an enlarged cross-sectional view of a portion of an example of a surgical reamer assembly.

FIG. 9 is an enlarged side view of a portion of an example of a surgical reamer assembly.

FIG. 10 is an enlarged cross-sectional view of a portion of an example of a surgical reamer assembly.

### DETAILED DESCRIPTION

Surgical reamer systems, including a ball hex driver and a reamer head with an internal hex housing, are beneficial as they provide a clinician with more flexibility in the operation of the reamer. The main reason there is more flexibility using a reamer system including a ball hex driver and a reamer head with an internal hex housing is that a longitudinal axis of the ball hex driver does not have to be aligned with a longitudinal of the reamer head. However, existing surgical reamer systems with ball hex drivers and reamer heads, including internal hex housings, rely on pressure from the ball hex driver to maintain the assembly. In other words, gravity can decouple the reamer head from the ball hex driver if the clinician removes the ball hex driver from the reamer head.

The flexibility provided by the ball hex driver and reamer head system helps in complex surgical procedures where precise angulation and positioning are required. The internal hex housing in the reamer head allows the ball hex driver to be inserted at various angles relative to the longitudinal axis of the reamer head. The adjustability helps in orthopedic surgeries involving hard-to-reach areas or requiring specific angular adjustments for optimal bone reaming. The reliance on the pressure exerted by the ball hex driver to maintain the assembly means that any reduction in this pressure, such as when the tool is oriented in a way that gravity assists in disengagement between the ball hex driver and the reamer head, can cause separation of the reamer head from the driver, potentially disrupting the surgical process.

The present disclosure includes one or more systems to address the detachment of the reamer head and the ball hex driver. In examples, a surgical reamer assembly can include a reamer head configured to ream a bone of a patient. The reamer head can include an internal hex housing. A ball hex driver can be configured to be inserted into the internal hex housing. The surgical reamer assembly can also include a retaining ring within the internal hex housing. The retaining ring can be configured to retain the ball hex driver within the reamer head. In examples, a surgical reamer assembly can include a reamer head and a retaining ring. The reamer head can include an internal hex housing, the internal hex housing configured to receive a ball hex driver. The retaining ring can be located within the internal hex housing. The retaining ring can be configured to expand and contract to permit the insertion and removal of the ball hex driver from the reamer head and retain the ball hex driver within the reamer head, respectively.

The examples described herein help solve the issue of unintentional detachment by incorporating a retaining ring within the internal hex housing of the reamer head. The retaining ring enhances the connection stability between the ball hex driver and the reamer head. The retaining ring is designed to mechanically engage with the ball hex driver, preventing it from slipping out due to gravity or other forces that might occur during surgery. The retaining ring is configured to expand and contract, allowing for easy insertion and removal of the ball hex driver and facilitating quick assembly and disassembly. Thus, the retaining ring helps maintain surgical efficiency and predictability, as it ensures that the reamer assembly remains securely connected throughout the procedure, yet can be quickly disassembled for cleaning and sterilization.

The above discussion is intended to provide an overview of the subject matter of the present patent application. It is not intended to provide an exclusive or exhaustive explanation of the invention. The description below provides further information about the present patent application.

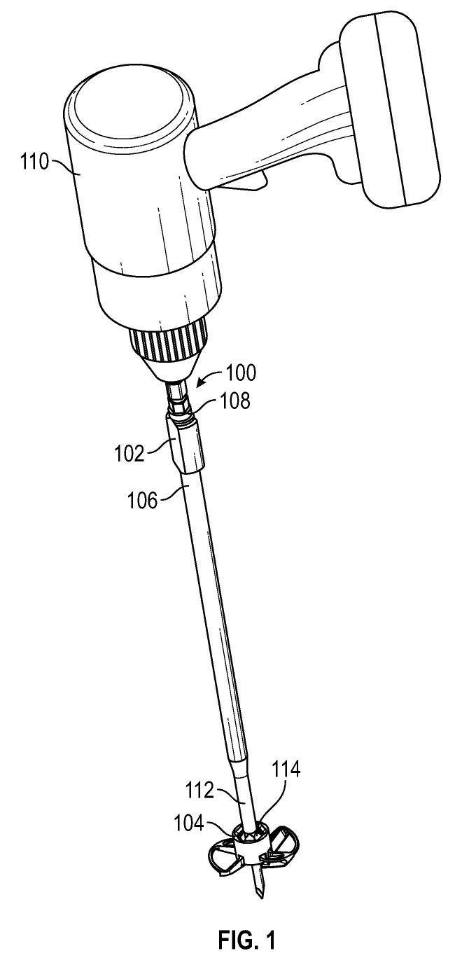

FIG. 1 is a perspective view of an example of a surgical reamer assembly 100. The surgical reamer assembly 100 can be configured to ream a bone of a patient. For example, the surgical reamer assembly 100 can be configured to ream a humerus of a patient to prepare the humerus to receive a prosthetic implant. The surgical reamer assembly 100 can include a ball hex driver to provide adjustability to a longitudinal axis of a ball hex driver relative to a longitudinal axis of a reamer body. In examples, the surgical reamer assembly 100 can include a ball hex driver 102 and a reamer head 104. The surgical reamer assembly 100 can be configured to be attached to a drill 110 to provide power to the surgical reamer assembly 100.

The ball hex driver 102 can be configured to be attached to the reamer head 104 and the drill 110 to transfer power from the drill 110 to the reamer head 104. The ball hex driver 102 can extend between a proximal section 106 and a distal section 112. The proximal section 106 can include an engagement interface 108 configured to attach the ball hex driver 102 to the drill 110. As shown in FIG. 1, the engagement interface 108 can be a hex shank system. In other examples, the engagement interface 108 can be an insert, twist, secure system, quick-change system, any other system used to attach a shaft to a drill, or the like. The distal section 112 can include an attachment interface 114 to attach the ball hex driver 102 to the reamer head 104. As shown in FIG. 1, the attachment interface 114 can include a ball hex end with a larger diameter than the average diameter of the shaft of the ball hex driver 102.

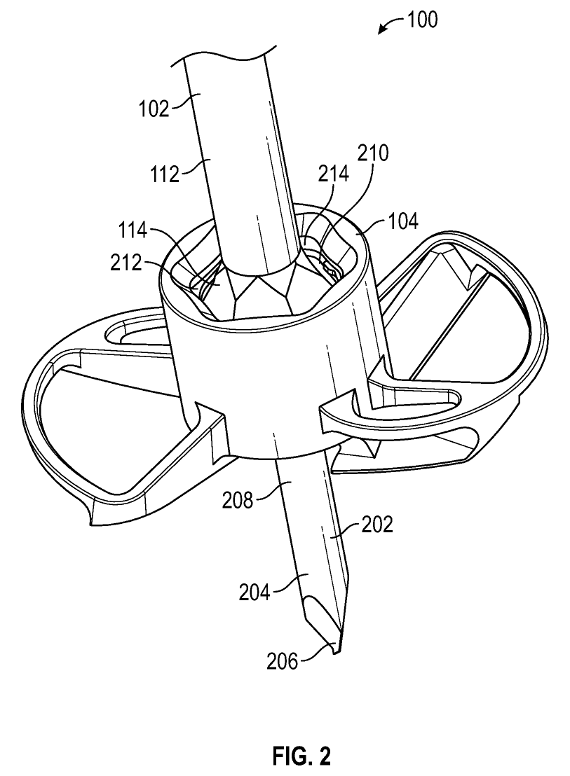

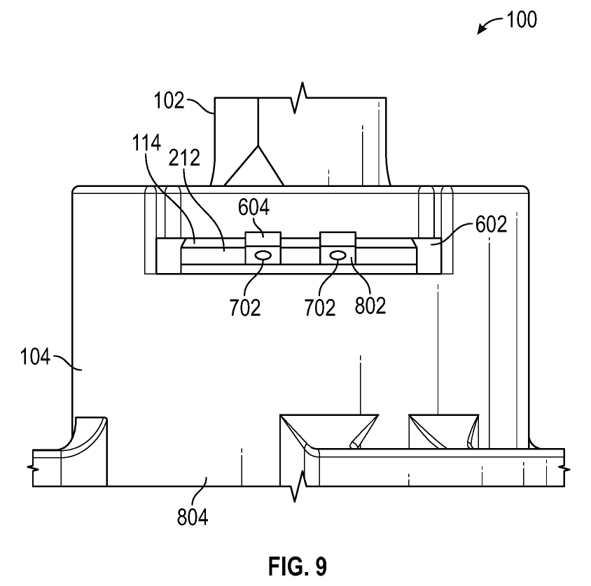

In examples, as will be discussed herein, the surgical reamer assembly 100 can include a retaining ring (e.g., a retaining ring 212 (first shown in FIG. 2)) configured to maintain attachment between the ball hex driver 102 and the reamer head 104. The retaining ring helps make the surgical reamer assembly 100 more adjustable such that a clinician can draw the surgical reamer assembly 100 away from the bone of the patient without the ball hex driver 102 detaching from the reamer head 104. The surgical reamer assembly 100 and the retaining ring 212 will be discussed in more detail herein with reference to FIG. 2 – FIG. 9.

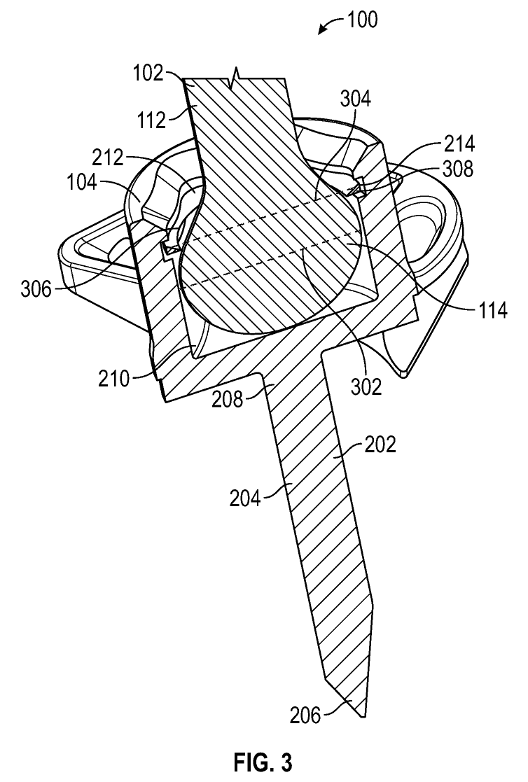

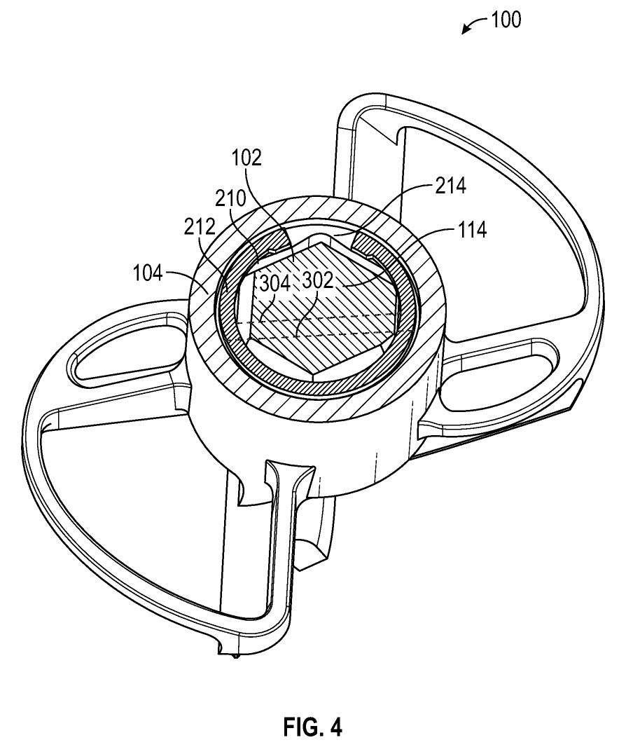

FIG. 2 – FIG. 4 will be discussed together below. FIG. 2 is an enlarged perspective view of a portion of an example of a surgical reamer assembly 100. FIG. 3 is an enlarged cross-sectional view of a portion of an example of a surgical reamer assembly 100. FIG. 4 is an enlarged cross-sectional view of a portion of an example of a surgical reamer assembly 100.

As first shown in FIG. 2, the surgical reamer assembly 100 can include the retaining ring 212. The reamer head 104 can include a center peg 202 extending between a distal portion 204 and a proximal portion 208. The distal portion 204 of the center peg 202 can include a pointed end 206. The pointed end 206 can be configured to help position the surgical reamer assembly 100 on the bone of the patient. The proximal portion 208 of the center peg 202 can be adjacent to the internal hex housing 210 formed within the reamer head 104.

The internal hex housing 210 can be configured to receive the attachment interface 114 of the ball hex driver 102. The attachment interface 114 and the internal hex housing 210 permit adjustability of the surgical reamer assembly 100 such that a longitudinal axis of the ball hex driver 102 can be misaligned with a longitudinal axis of the reamer head 104 during operation of the surgical reamer assembly 100.

The retaining ring 212 can hold the ball hex driver 102 and the reamer head 104 together. The retaining ring 212 can move between a retracted and expanded configuration to retain the ball hex driver 102 within the reamer head 104 or to permit the installation or removal of the ball hex driver 102 and the reamer head 104, respectively. In examples, the retaining ring 212 can be a c-ring, o-ring, u-ring, other retaining rings used to hold a shaft in a cavity, or the like. The retaining ring 212 can include steel, aluminum, copper, gold, titanium, composites, polymers, monomers, alloys or composites thereof, or the like.

The retaining ring groove 214 can be formed within the reamer head 104. For example, the retaining ring groove 214 can be formed in the internal hex housing 210. In another example, the retaining ring groove 214 can be formed in the reamer head 104 adjacent to the internal hex housing 210. The retaining ring groove 214 can be configured to hold the retaining ring 212 within the reamer head 104. The retaining ring groove 214 can be sized such that the retaining ring 212 can move between the expanded or retracted positions while installed within the retaining ring groove 214 (as shown in FIG. 3). The retaining ring groove 214 can extend around an entirety of the reamer head 104, or at least a portion of the reamer head 104.

Now, referring to FIG. 3 and FIG. 4. The retaining ring 212 can include a tapered surface 306 and a flat surface 308. The tapered surface 306 can be opposite the retaining ring 212 from the flat surface 308. The tapered surface 306 can be configured to guide the ball hex driver 102 into the reamer head 104. Specifically, the tapered surface 306 can be configured to guide the attachment interface 114 into the internal hex housing 210. The flat surface 308 can be configured to engage with the attachment interface 114 to retain the ball hex driver 102 within the reamer head 104. More specifically, the tapered surface 306 of the retaining ring 212 can engage with the retaining ring groove 214 as the attachment interface 114 engages with the flat surface 308 to hold the ball hex driver 102 within the reamer head 104. To enable the engagement between the flat surface 308 and the attachment interface 114, the ball hex driver diameter 302 can be larger than the retaining ring diameter 304 in a contracted configuration (as shown in FIG. 3 and FIG. 4).

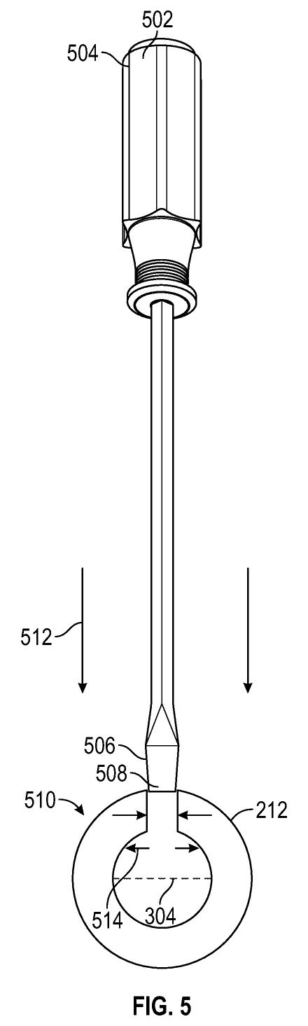

FIG. 5 is a schematic illustration of an example removal tool 502 and an example retaining ring 212. The removal tool 502 can be configured to be used to move the retaining ring 212 (e.g., c-ring 510) between the contracted and expanded configurations to change the retaining ring diameter 304. The removal tool 502 can include a ratchet, needle nose ends, pliers, screwdriver, or any other tool that can be used to manipulate the retaining ring 212 to facilitate the assembly and disassembly of the surgical reamer assembly 100. The removal tool 502 can extend between a proximal portion 504 and a distal portion 506. The proximal portion 504 can include a handle to help a clinician hold the removal tool 502 to engage with the retaining ring 212 during the removal of the ball hex driver 102 (first shown in FIG. 1) from the reamer head 104 (first shown in FIG. 1).

The distal portion 506 can include a tapered profile 508. The tapered profile 508 can be configured to further spread the retaining ring 212 in the second direction 514 as the removal tool 502 is moved into the retaining ring 212 in the first direction 512. The tapered profile 508 increases the movement of the retaining ring 212 in the second direction 514 as the removal tool 502 is moved more in the first direction 512. In other words, as the removal tool 502 first engages with the retaining ring 212, the amount of spread is minimal. As the removal tool 502 is moved in the first direction 512, the tapered profile 508 increases a spread of the retaining ring 212 the further the removal tool 502 is moved in the first direction 512.

As shown in FIG. 5, the removal tool 502 can be an implement including a tapered profile 508. In other examples, the removal tool can be a needle nose plier, snap ring remover tool, standard plier, or other types of tools that can be used to expand the retaining ring 212 to permit the installation or removal of the ball hex driver 102 from the reamer head 104.

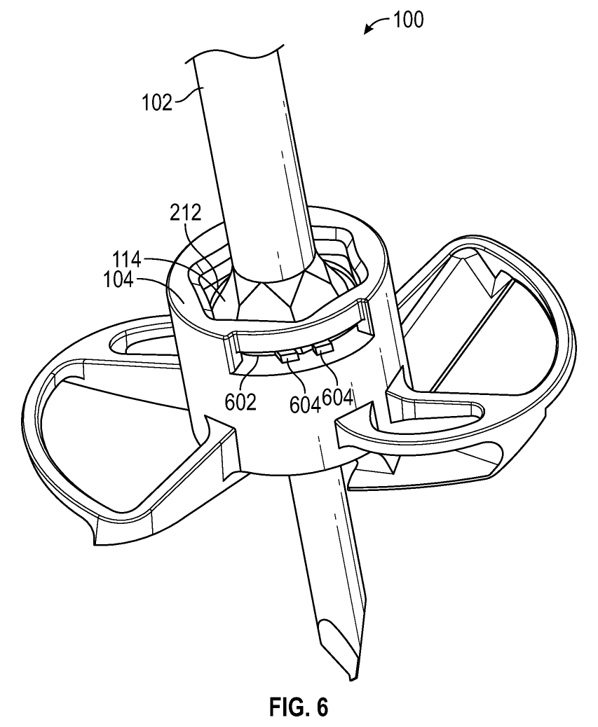

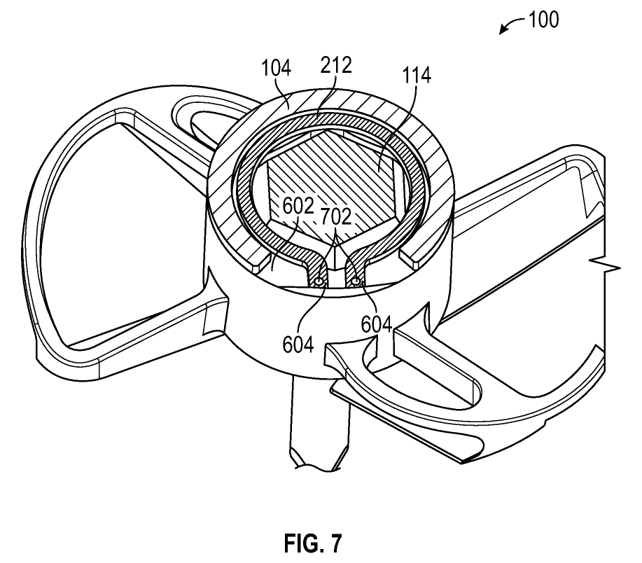

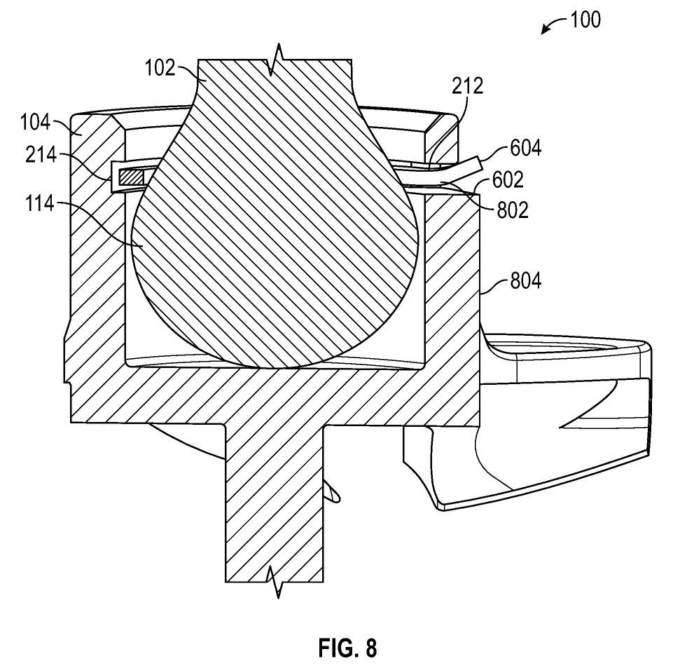

FIG. 6 – FIG. 9 will be discussed together below. FIG. 6 is an enlarged perspective view of a portion of an example of a surgical reamer assembly 100. FIG. 7 is an enlarged cross-sectional view of a portion of an example of a surgical reamer assembly 100. FIG. 8 is an enlarged cross-sectional view of a portion of an example of a surgical reamer assembly 100. FIG. 9 is an enlarged side view of a portion of an example of a surgical reamer assembly 100. As first shown in FIG. 6, the reamer head 104 can include a retaining ring engagement window 602 and the retaining ring 212 can include engagement tabs 604.

The retaining ring engagement window 602 can be configured to provide access to the engagement tabs 604 of the retaining ring 212. The retaining ring engagement window 602 can be configured to provide access to different types of tools, such as tapered edges, rounded edges, varying widths, lengths, depths, or the like. The retaining ring engagement window 602 can extend around at least a portion of a periphery 804 (FIG. 8) of the reamer head 104 to the retaining ring groove 214. The amount of the periphery 804 that the retaining ring engagement window 602 extends around the reamer head 104 can be varied based on the application of the surgical reamer assembly 100. For example, if access is easier, the retaining ring engagement window 602 can extend less around the periphery 804 of the reamer head 104. If the clinician needs more access points because access is more difficult, the retaining ring engagement window 602 can extend around more of the periphery 804 of the reamer head 104.

In examples, the engagement tabs 604 can extend radially outward from the retaining ring 212. The engagement tabs can be configured to extend at least partially into the retaining ring engagement window. For example, the engagement tab 604 can extend far enough into the retaining ring engagement window 602 to provide access to the tabs so the clinician can move the retaining ring 212 into the expanded configuration or guide the retaining ring 212 back to the contracted position. In examples, the engagement tab 604 can be configured to be grabbed by pliers, wrenches, or the like. In other words, the engagement tabs 604 can be configured to provide a user of the surgical reamer assembly 100 better access to the retaining ring 212 during assembly or disassembly of the surgical reamer assembly 100.

Each engagement tab 604 of the retaining ring 212 can include an aperture 702 (first shown in FIG. 7). The aperture 702 can be configured to receive a tool (e.g., the removal tool 502) to move the retaining ring 212 between the expanded and retracted configurations. For example, the removal tool can be a snap-ring removal tool, needle nose pliers, another tool that can engage with the aperture 702 to adjust the retaining ring 212, or the like. The aperture 702 can extend through the engagement tab 604 such that the aperture 702 can be reached from either direction (e.g., from a distal or proximal surface). The aperture 702 can be sized based on the type of removal tool being used. For example, if the removal tool is a snap ring removal tool having a larger distal tip, the aperture 702 can be made larger.

As shown in FIG. 8 and FIG. 9, the engagement tabs 604 can include a bend 802. The bend 802 can provide clearance between the engagement tabs 604 and the retaining ring engagement window 602 as the engagement tabs 604 extend toward a periphery 804 of the reamer head 104. The additional clearance can help with engagement between the engagement tabs 604 and a tool (e.g., removal tool 502) to help move the engagement tabs 604 toward the expanded configuration during assembly and disassembly of the ball hex driver 102 and the reamer head 104.

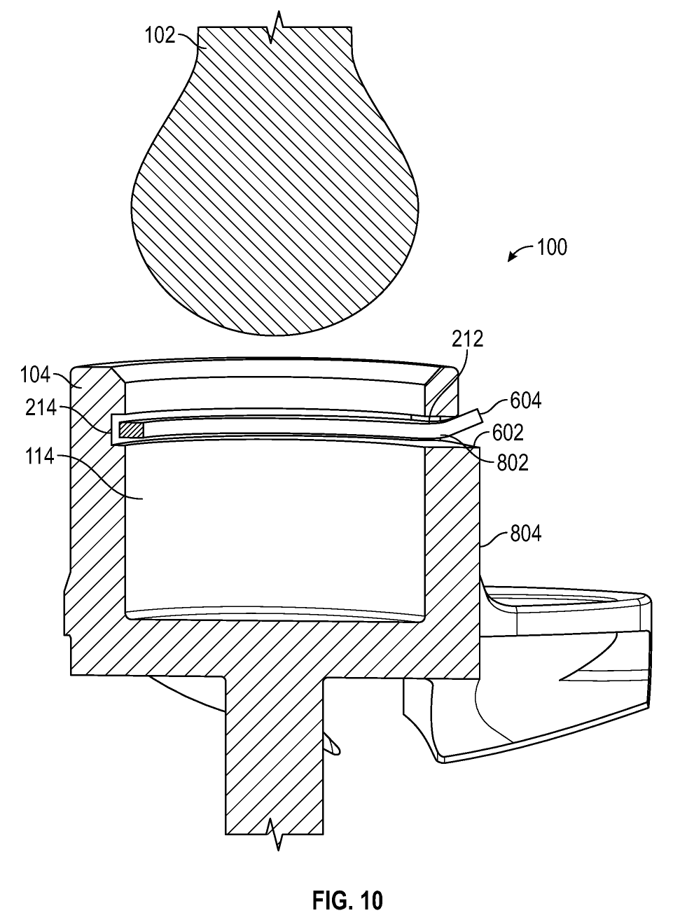

FIG. 10 is an enlarged cross-sectional view of a portion of an example of a surgical reamer assembly 100. As shown in FIG. 10, when the ball hex driver 102 is removed from the reamer head 104 (e.g., either before installation of the ball hex driver 102 into the reamer head 104 or after removal of the ball hex driver 102 from the reamer head 104) the retaining ring 212 remains within the retaining ring groove 214 of the reamer head 104.

The following non-limiting examples detail certain aspects of the present subject matter that solve the challenges and provide the benefits discussed herein, among other things.

Example 1 is a surgical reamer assembly comprising: a reamer head configured to ream a bone of a patient, the reamer head including: an internal hex housing; a ball hex driver insertable into the internal hex housing; and a retaining ring located within the internal hex housing, the retaining ring configured to retain the ball hex driver within the reamer head.

In Example 2, the subject matter of Example 1 optionally includes wherein the retaining ring comprises a tapered surface configured to guide the ball hex driver into the internal hex housing.

In Example 3, the subject matter of Example 2 optionally includes wherein the retaining ring comprises a flat surface opposite the tapered surface to help retain the ball hex driver within the internal hex housing, and wherein a contracted inner diameter of the retaining ring is smaller than a diameter of the ball hex driver such that the flat surface engages the ball hex driver.

In Example 4, the subject matter of any one or more of Examples 1–3 optionally include wherein the reamer head comprises: a center peg including: a proximal portion, the center peg extending between a distal portion and the proximal portion, the proximal portion including the internal hex housing.

In Example 5, the subject matter of Example 4 optionally includes wherein the ball hex driver comprises: a proximal section including: an engagement interface configured to attach to a drill; and a distal section, the ball hex driver extending between the proximal section and the distal section, the distal section including: an attachment interface configured to be inserted within the internal hex housing and engage with the retaining ring to hold the ball hex driver within the internal hex housing.

In Example 6, the subject matter of any one or more of Examples 1–5 optionally include wherein the reamer head comprises: a retaining ring groove configured to hold the retaining ring within the reamer head.

In Example 7, the subject matter of Example 6 optionally includes wherein the reamer head comprises: a retaining ring engagement window extending from a periphery of the reamer head to the retaining ring groove.

In Example 8, the subject matter of Example 7 optionally includes wherein the retaining ring is a c-ring including a tapered profile configured to guide the ball hex driver into the internal hex housing.

In Example 9, the subject matter of Example 8 optionally includes wherein the retaining ring comprises: engagement tabs extending radially outward from the retaining ring, the engagement tabs configured to extend at least partially into the retaining ring engagement window, the engagement tabs configured to provide a user of the surgical reamer assembly better access to the retaining ring during disassembly of the surgical reamer assembly.

In Example 10, the subject matter of Example 9 optionally includes wherein each engagement tab of the engagement tabs includes: an aperture extending through the engagement tab, the aperture configured to further improve access to the retaining ring during disassembly of the surgical reamer assembly.

In Example 11, the subject matter of Example 10 optionally includes wherein at least one engagement tab of the engagement tabs includes: a bend configured to provide clearance between the engagement tab and the retaining ring engagement window as the engagement tab extends toward a periphery of the reamer head.

Example 12 is a surgical reamer assembly comprising: a reamer head including: an internal hex housing, the internal hex housing configured to receive a ball hex driver; and a retaining ring located within the internal hex housing, the retaining ring configured to expand and contract to permit insertion and removal of the ball hex driver from the reamer head and retain the ball hex driver within the reamer head, respectively.

In Example 13, the subject matter of Example 12 optionally includes wherein the retaining ring comprises a tapered surface configured to guide the ball hex driver into the internal hex housing.

In Example 14, the subject matter of Example 13 optionally includes wherein the retaining ring comprises a flat surface opposite the tapered surface to help retain the ball hex driver within the internal hex housing.

In Example 15, the subject matter of any one or more of Examples 12–14 optionally include wherein the reamer head comprises: a center peg including: a distal portion including a pointed end, the pointed end configured to help position the surgical reamer assembly on a bone of a patient; and a proximal portion, the center peg extending between the distal portion and the proximal portion, the proximal portion including the internal hex housing.

In Example 16, the subject matter of any one or more of Examples 12–15 optionally include wherein the reamer head comprises: a retaining ring groove configured to hold the retaining ring within the reamer head.

In Example 17, the subject matter of Example 16 optionally includes wherein the reamer head comprises: a retaining ring engagement window extending from a periphery of the reamer head to the retaining ring groove.

In Example 18, the subject matter of Example 17 optionally includes wherein the retaining ring is a c-ring including a tapered profile configured to guide the ball hex driver into the internal hex housing.

In Example 19, the subject matter of Example 18 optionally includes wherein the retaining ring comprises: engagement tabs extending radially outward from the retaining ring, the engagement tabs configured to extend at least partially into the retaining ring engagement window, the engagement tabs configured to provide a user of the surgical reamer assembly better access to the retaining ring during disassembly of the surgical reamer assembly.

In Example 20, the subject matter of Example 19 optionally includes wherein each engagement tab of the engagement tabs includes: an aperture extending through the engagement tab, the aperture configured to further improve access to the retaining ring during disassembly of the surgical reamer assembly.

Example 21 includes a method, apparatus, or system including any element of any of Examples 1-20.

The above-detailed description includes references to the accompanying drawings, which form a part of the detailed description. The drawings show, by way of illustration, specific examples that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other examples may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the examples should be determined with reference to the appended claims, along with the full scope of equivalents to which such claims are entitled.

What is claimed is:

1. A surgical reamer assembly comprising:
a reamer head configured to ream a bone of a patient, the reamer head including:
an internal hex housing;
a ball hex driver insertable into the internal hex housing; and
a retaining ring located within the internal hex housing, the retaining ring configured to retain the ball hex driver within the reamer head.

2. The surgical reamer assembly of claim 1, wherein the retaining ring comprises a tapered surface configured to guide the ball hex driver into the internal hex housing.

3. The surgical reamer assembly of claim 2, wherein the retaining ring comprises a flat surface opposite the tapered surface to help retain the ball hex driver within the internal hex housing, and wherein a contracted inner diameter of the retaining ring is smaller than a diameter of the ball hex driver such that the flat surface engages the ball hex driver.

4. The surgical reamer assembly of claim 1, wherein the reamer head comprises:
a center peg including:
a distal portion including a pointed end, the pointed end configured to help position the surgical reamer assembly on a bone of a patient; and
a proximal portion, the center peg extending between the distal portion and the proximal portion, the proximal portion including the internal hex housing.

5. The surgical reamer assembly of claim 4, wherein the ball hex driver comprises:
a proximal section including:
an engagement interface configured to attach to a drill; and
a distal section, the ball hex driver extending between the proximal section and the distal section, the distal section including:
an attachment interface configured to be inserted within the internal hex housing and engage with the retaining ring to hold the ball hex driver within the internal hex housing.

6. The surgical reamer assembly of claim 1, wherein the reamer head comprises:
a retaining ring groove configured to hold the retaining ring within the reamer head.

7. The surgical reamer assembly of claim 6, wherein the reamer head comprises:
a retaining ring engagement window extending from a periphery of the reamer head to the retaining ring groove.

8. The surgical reamer assembly of claim 7, wherein the retaining ring is a c-ring including a tapered profile configured to guide the ball hex driver into the internal hex housing.

9. The surgical reamer assembly of claim 8, wherein the retaining ring comprises:
engagement tabs extending radially outward from the retaining ring, the engagement tabs configured to extend at least partially into the retaining ring engagement window, the engagement tabs configured to provide a user of the surgical reamer assembly better access to the retaining ring during disassembly of the surgical reamer assembly.

10. The surgical reamer assembly of claim 9, wherein each engagement tab of the engagement tabs includes:
an aperture extending through the engagement tab, the aperture configured to further improve access to the retaining ring during disassembly of the surgical reamer assembly.

11. The surgical reamer assembly of claim 10, wherein at least one engagement tab of the engagement tabs includes:
a bend configured to provide clearance between the engagement tab and the retaining ring engagement window as the engagement tab extends toward a periphery of the reamer head.

12. A surgical reamer assembly comprising:
a reamer head including:
an internal hex housing, the internal hex housing configured to receive a ball hex driver; and
a retaining ring located within the internal hex housing, the retaining ring configured to expand and contract to permit insertion and removal of the ball hex driver from the reamer head and retain the ball hex driver within the reamer head, respectively.

13. The surgical reamer assembly of claim 12, wherein the retaining ring comprises a tapered surface configured to guide the ball hex driver into the internal hex housing.

14. The surgical reamer assembly of claim 13, wherein the retaining ring comprises a flat surface opposite the tapered surface to help retain the ball hex driver within the internal hex housing.

15. The surgical reamer assembly of claim 12, wherein the reamer head comprises:
a center peg including:
a proximal portion, the center peg extending between a distal portion and the proximal portion, the proximal portion including the internal hex housing.

16. The surgical reamer assembly of claim 12, wherein the reamer head comprises:
a retaining ring groove configured to hold the retaining ring within the reamer head.

17. The surgical reamer assembly of claim 16, wherein the reamer head comprises:
a retaining ring engagement window extending from a periphery of the reamer head to the retaining ring groove.

18. The surgical reamer assembly of claim 17, wherein the retaining ring is a c-ring including a tapered profile configured to guide the ball hex driver into the internal hex housing.

19. The surgical reamer assembly of claim 18, wherein the retaining ring comprises:
engagement tabs extending radially outward from the retaining ring, the engagement tabs configured to extend at least partially into the retaining ring engagement window, the engagement tabs configured to provide a user of the surgical reamer assembly better access to the retaining ring during disassembly of the surgical reamer assembly.

20. The surgical reamer assembly of claim 19, wherein each engagement tab of the engagement tabs includes:
an aperture extending through the engagement tab, the aperture configured to further improve access to the retaining ring during disassembly of the surgical reamer assembly.
